(19) United States
(12) Patent Application Publication
(10) Pub. No.: US 2009/0035395 A1
(43) Pub. Date: Feb. 5, 2009

(54) SPONDIAS MOMBIN L. EXTRACT AND METHODS OF EXTRACTING AND USING SUCH EXTRACT

(76) Inventors: Peter KOEPKE, Cold Spring, NY (US); Ven SUBBIAH, Garner, NC (US); Matthew E. BUROW, Slidell, LA (US)

Correspondence Address: COX SMITH MATTHEWS INCORPORATED, 112 EAST PECAN STREET, SUITE 1800, SAN ANTONIO, TX, 78205-1521, US

(21) Appl. No.: 12/032,987

(22) Filed: Feb. 18, 2008

Related U.S. Application Data
(60) Provisional application No. 60/953,322, filed on Aug. 1, 2007.

(51) Int. Cl. A61K 36/00; A61K 31/045; A61K 31/22; A61K 31/01; A61P 35/00; A61P 29/00

(52) U.S. Cl. 424/725; 514/724; 514/546; 514/762

(57) ABSTRACT

A method of inhibiting COX-2, inhibiting NF-Kappa B activation, treating inflammation, or treating cancer may comprise administering a therapeutically effective amount of an extract of Spondias mombin L. to a patient. A medicament as described herein may comprise a pharmaceutically acceptable vehicle and a therapeutically effective amount of an extract of Spondias mombin L. suspended in the vehicle. A method of making an extract of Spondias mombin L. may comprise creating a component solution by treating Spondias mombin L. material with an extractor and a solvent and producing an extract by at least partially removing liquid from the component solution. An extract of Spondias mombin L. may comprise components extracted using various solvents.

### CROSS REFERENCE TO RELATED APPLICATIONS

This application claims priority to U.S. Provisional Application No. 60/953,322, filed on Aug. 1, 2007, the disclosure of which is incorporated herein by reference.

### FIELD

This application relates generally to plant extracts for treating inflammation and cancer.

### BACKGROUND

Rheumatoid arthritis is a chronic inflammatory disease affecting multiple tissues, but typically producing its most pronounced symptoms in the joints. It is progressive, degenerative and ultimately debilitating. The chronic inflammation in joints leads to the destruction of the soft tissue, the synovium and cartilage, as well as erosion of the articular surfaces of bones. The disease is estimated to affect over 3.2 million people in the United States, Europe and Japan. It is more prevalent in women, who are estimated to account for a majority of the cases.

Inflammation is a natural defense of the body to protect against foreign substances or injury, but it can cause problems in certain diseases. Inappropriate inflammation can be treated with traditional steroids, like the glucocorticoid cortisol, therapeutic proteins produced by recombinant DNA technology, and/or non-steroidal anti-inflammatory drugs (NSAIDs).

Prostaglandins are a family of chemicals that are produced by the cells of the body and serve many essential functions including the promotion of pain, inflammation, and fever. Additionally, some prostaglandins support the function of platelets, necessary for blood clotting, and protect the stomach lining from the damaging effects of acid. Prostaglandins are produced within the body's cells by the enzyme cyclooxygenase-2 (COX-2).

COX-2 is an enzyme involved in many functions, including but not limited to inducing pain. COX-2 is located specifically in areas of the body that are responsible for inflammation and not in the stomach. COX-2 is active in our bodies, ideally on a limited basis; however, factors such as diet, stress and injury can increase COX-2 activity. When COX-2 is active on a continual basis, constant pain ensues.

Even though the specific mechanism of action is not completely understood, it has been found that inhibiting COX-2 results in the apoptosis of cancer cells. See Johnsen, et al., “Cyclooxygenase-2 Is Expressed in Neuroblastoma, and Nonsteroidal Anti-Inflammatory Drugs Induce Apoptosis and Inhibit Tumor Growth In Vivo,” Cancer Research; Vol. 64, pages. 7210-7215 (Oct. 15, 2004); and Lau, et al., “Cyclooxygenase inhibitors modulate the p53/hdm2 pathway and enhance chemotherapy-induced apoptosis in neuroblastoma,” Oncogene, Vol. 26, pages 1920-1931 (2007).

Therefore, plant extracts that may inhibit COX-2 may treat various diseases, including but not limited to inflammation, arthritis, muscle pain, and cancer.

### SUMMARY

A method of inhibiting COX-2, inhibiting NF-Kappa B activation, treating inflammation, or treating cancer may comprise administering a therapeutically effective amount of an extract of Spondias mombin L. to a patient. A medicament as described herein may comprise a pharmaceutically acceptable vehicle and a therapeutically effective amount of an extract of Spondias mombin L. suspended in the vehicle. A method of making an extract of Spondias mombin L. may comprise creating a component solution by treating Spondias mombin L. material with an extractor and a solvent and producing an extract by at least partially removing liquid from the component solution. An extract of Spondias mombin L. may comprise components extracted using various solvents.

### BRIEF DESCRIPTION OF THE DRAWINGS

FIG. 1 is a graph that illustrates the results from an experiment to test the inhibition of COX-2 in vitro by an extract of Spondias mombin L. at a concentration of 9 μg/ml, extracted using four different solvents. The graph in FIG. 1 comprises percentage inhibition of human recombinant COX-2 on the y-axis, and the solvent used to extract components from the material of Spondias mombin L. on the x-axis.

FIG. 2 is a graph that illustrates results from a replicate of the experiment of FIG. 1. The graph in FIG. 2 comprises percentage inhibition of human recombinant COX-2 on the y-axis, and the solvent used to extract components from the material of Spondias mombin L. on the x-axis.

FIG. 3 is a graph that illustrates results from another replicate of the experiment of FIG. 1. The graph in FIG. 3 comprises percentage inhibition of human recombinant COX-2 on the y-axis, and the solvent used to extract components from the material of Spondias mombin L. on the x-axis.

FIG. 4 is a graph that illustrates the results from yet another replicate of the experiment of FIG. 1. The graph in FIG. 4 comprises percentage inhibition of human recombinant COX-2 on the y-axis, and the solvent used to extract components from the material of Spondias mombin L. on the x-axis.

FIG. 5 is a graph that illustrates the results from an experiment to test the inhibition of COX-2 in vitro by a methanol extract of Spondias mombin L. at varying concentrations. The graph in FIG. 5 comprises percentage inhibition of human recombinant COX-2 on the y-axis, and concentration of the methanol extract of Spondias mombin L. on the x-axis.

FIG. 6 is a graph that illustrates results from a replicate of the experiment of FIG. 5. The graph in FIG. 6 comprises percentage inhibition of human recombinant COX-2 on the y-axis, and concentration of a methanol extract of Spondias mombin L. on the x-axis.

FIG. 7 is a graph that illustrates the results of an experiment to test the decrease in cell proliferation of SK-Mel 28 human melanoma cells, cultured in media with serum, caused by a methanol extract of Spondias mombin L. at varying concentrations. The graph in FIG. 7 comprises percentage decrease in cell proliferation on the y-axis, and concentration of a methanol extract of Spondias mombin L. on the x-axis.

FIG. 8 is a graph that illustrates results of an experiment similar to the experiment of FIG. 7, except that the SK-Mel 28 human melanoma cells were cultured in media without serum. The graph in FIG. 8 comprises percentage decrease in cell proliferation on the y-axis, and concentration of a methanol extract of Spondias mombin L. on the x-axis.

FIG. 9 is a graph that illustrates the results from an experiment to test the inhibition of COX-2 in vitro by fractions of a methanol extract of Spondias mombin L., at a concentration of 10 g/ml, with percentage inhibition of human recombinant COX-2 on the y-axis, and fractions identified by elution time in minutes on the x-axis.

FIGS. 10A-10D are high pressure liquid chromatography profiles based on the liquid chromatography-mass spectrometry and mass detection pertaining to fractions 1 and 2 in FIG. 9 of the methanol extract of Spondias mombin L.

FIG. 11 is a graph that illustrates the results from an experiment to test the cell colony number of three breast cancer cell lines treated with an ethyl-acetate extract of Spondias mombin L. The graph in FIG. 11 comprises cell line on the x-axis and percent cell colony count normalized with the control to 100% on the y-axis.

FIG. 12 is a graph that illustrates the results from an experiment to test the suppression of tumor growth (MCF-7 cells) in nude mice by an ethyl-acetate extract of Spondias mombin L. administered intraperitoneally at a concentration of 8 mg/ml. The graph in FIG. 12 comprises normalized tumor volume on the y-axis and day post-injection of MCF-7 cells on the x-axis.

FIG. 13 is a graph that illustrates the results of an experiment to test NF-Kappa B activation in human embryonic kidney 293 cells exposed to a methanol extract of Spondias mombin L. at varying concentrations and a known activator of NF-Kappa B, namely PMA. The graph in FIG. 13 comprises treatment group on the x-axis and NF-Kappa B activation normalized to the control (with no methanol extract) to 100%.

FIG. 14 is a graph that illustrates the results of an experiment to test NF-Kappa B activation in human embryonic kidney 293 cells exposed to a methanol extract of Spondias mombin L. at varying concentrations and a known activator of NF-Kappa B, namely TNF. The graph in FIG. 14 comprises treatment group on the x-axis and NF-Kappa B activation normalized to the control (with no methanol extract) to 100%.

FIG. 15 is a graph that illustrates the results of an experiment to test NF-Kappa B activation in human embryonic kidney 293 cells exposed to an aqueous extract of Spondias mombin L. at varying concentrations and a known activator of NF-Kappa B, namely PMA. The graph in FIG. 15 comprises treatment group on the x-axis and NF-Kappa B activation normalized to the control (with no aqueous extract) to 100%.

FIG. 16 is a graph that illustrates the results of an experiment to test NF-Kappa B activation in human embryonic kidney 293 cells exposed to an aqueous extract of Spondias mombin L. at varying concentrations and a known activator of NF-Kappa B, namely TNF. The graph in FIG. 16 comprises treatment group on the x-axis and NF-Kappa B activation normalized to the control (with no aqueous extract) to 100%.

FIG. 17 is a graph that illustrates the results of an experiment to test NF-Kappa B activation in human embryonic kidney 293 cells exposed to a hexane extract of Spondias mombin L. at varying concentrations and a known activator of NF-Kappa B, namely TNF. The graph in FIG. 17 comprises treatment group on the x-axis and NF-Kappa B activation normalized to the control (with no hexane extract) to 100%.

FIG. 18 is a graph that illustrates the results of an experiment to test NF-Kappa B activation in human embryonic kidney 293 cells exposed to a hexane extract of Spondias mombin L. at varying concentrations and a known activator of NF-Kappa B, namely PMA. The graph in FIG. 18 comprises treatment group on the x-axis and NF-Kappa B activation normalized to the control (with no hexane extract) to 100%.

FIG. 19 is a graph that illustrates results from a replicate of the experiment of FIG. 14, namely an experiment to test NF-Kappa B activation in human embryonic kidney 293 cells exposed to a methanol extract of Spondias mombin L. at varying concentrations and a known activator of NF-Kappa B, namely TNF. The graph in FIG. 19 comprises treatment group on the x-axis and NF-Kappa B activation normalized to the control (with no methanol extract) to 100%.

FIG. 20 is a graph that illustrates results from a replicate of the experiment of FIG. 13, namely an experiment to test NF-Kappa B activation in human embryonic kidney 293 cells exposed to a methanol extract of Spondias mombin L. at varying concentrations and a known activator of NF-Kappa B, namely PMA. The graph in FIG. 20 comprises treatment group on the x-axis and NF-Kappa B activation normalized to the control (with no methanol extract) to 100%.

FIG. 21 is a graph that illustrates the results from an experiment to test the suppression of tumor growth (MDA-MB-231 cells) in nude mice by an ethyl-acetate extract of Spondias mombin L. administered intraperitoneally at a concentration of 8 mg/ml. The graph in FIG. 21 comprises normalized tumor volume on the y-axis and day post-injection of MDA-MB-231 cells on the x-axis.

### DETAILED DESCRIPTION

As used herein, the following terms should be understood to have the indicated meanings:

When an item is introduced by “a” or “an,” it should be understood to mean one or more of that item.

“Component” means any gas, liquid or solid of a molecule, chemical, macromolecule, compound, or element, alone or in combination.

“Component solution” means a mixture of one or more components contained, suspended, held, or dispersed in a liquid, solid, or gas.

“Comprises” means includes but is not limited to.

“Comprising” means including but not limited to.

“Condition” means a particular state of health, such as but not limited to a disordered or incorrectly functioning organ, part, structure or system of the body, an illness, a sickness, an ailment, a disease, a physical or mental suffering, a physical or mental distress, a physical or mental sensation, a physical or mental torment, or a physical or mental pain. A condition may include cancer or inflammation.

“COX-2” means cyclooxygenase-2.

“Extractor” means an apparatus, machine, instrument, tool, or combination thereof having at least one flask adaptable to contain a solvent or solution, at least one chamber adaptable to contain a material, and at least one condenser in fluid communication with a chamber and a flask. An extractor may have a funnel adaptable to recover the solvent at some point during the extraction process. A thimble may be used in connection with an extractor. A filter may be used in connection with an extractor. An extractor may be adaptable to be subjected to heat while not decreasing the integrity of the extractor. An extractor includes, but is not limited to, a Soxhlet extractor, as invented by Franz von Soxhlet in or around 1879, and several commercially available extractors such as, but not limited to, a Soxtherm™ extractor from Gerhardt GmbH, and Soxtec Systems™, which are automated or semi-automatic extractors made by FOSS™.

“Grind” means to reduce or lessen into relatively smaller particles or pieces by pulverizing, pounding, cutting, crushing, grating, rubbing harshly, carving, sawing, trimming, or dissolving an object, or a combination thereof.

“Having” means including but not limited to.

“IC50” means, with respect to a compound or formulation, the concentration of the compound or formulation that produces a 50% inhibition of COX-2.

“Inhibit” means to at least partially decrease the activity of an enzyme.

“Material” means any part of a plant including, but not limited to, bark, stem, leaf, bud, stalk, root, flower, pollen, branch, shoot, fruit, slip, vegetable, seed, or a combination thereof.

“Parenteral” means a type of route of administration of a component to a patient wherein the desired effect is systemic. Parenteral includes, but is not limited to, administering a component to a patient by injection or infusion, where such injection or infusion is intravenous, intraarterial, intramuscular, intracardiac, subcutaneous, intraosseous, intradermal, intraperitoneal, transdermal, transmucosal, inhalational, or a combination thereof.

“Patient” means a human or any other mammal.

“Pharmaceutically acceptable vehicle” means a carrier, diluent, adjuvant, or excipient, or a combination thereof, with which a component is administered to a patient. A pharmaceutically acceptable vehicle may include, but is not limited to, polyethylene glycol; wax; lactose; glucose; sucrose; magnesium stearate; silicic derivatives; calcium sulfate; dicalcium phosphate; starch; cellulose derivatives; gelatin; natural and synthetic gums such as, but not limited to, sodium alginate, polyethylene glycol and wax; suitable oil; saline; sugar solution such as, but not limited to, aqueous dextrose or aqueous glucose; DMSO; glycols such as, but not limited to, polyethylene or polypropylene glycol; lubricants such as, but not limited to, sodium oleate, sodium acetate, sodium stearate, sodium chloride, sodium benzoate, talc, and magnesium stearate; disintegrating agents, including calcium carbonate, sodium bicarbonate, agar, starch, and xanthan gum; and absorptive carriers such as, but not limited to, bentonite and klonin.

“Solvent” means a liquid or gas that has the ability to suspend, take out, draw out, separate, or attract one or more components to form a solution.

“Therapeutically effective amount” means an amount of a component that is sufficient to at least partially effect a treatment of a condition when administered to a patient. The therapeutically effective amount may vary depending on the condition, the route of administration of the component, and the age, weight, etc. of the patient being treated.

“Treat” means, with respect to a condition, to at least partially reduce, relieve, or alleviate any symptoms of the condition, to delay the onset of the condition or symptoms of the condition, to at least partially cure any symptom of the condition, or to at least partially prevent or inhibit the condition or a symptom of the condition, or a combination thereof, even if not discernible by the patient.

Spondias mombin L. is a plant in the family Anacardiaceae Lindley that typically grows in Central America, including but not limited to in tropical areas of Central America and in the Caribbean Islands. Common names for Spondias mombin L. include, but are not limited to, yellow mombin, caja fruit, jobo, and yellow Spanish plum. An extract of Spondias mombin L. that inhibits COX-2 may be made using the methods described herein. The extraction methods involve the use of solvents to extract components of Spondias mombin L. that at least partially inhibit COX-2. An extract as described herein may be used to treat inflammation in a patient. Alternatively, an extract as described herein may be used to treat cancer in a patient. It is well understood by persons of ordinary skill in the art that inhibiting COX-2 decreases inflammation and contributes to the apoptosis or a decrease in the proliferation of cancer cells in humans.

An extract of Spondias mombin L. may be made as follows. Material from Spondias mombin L. may be obtained, dried and ground. Alternatively, material from Spondias mombin L. may be ground into small pieces and then dried. The material may be dried in an oven such as but not limited to a drying oven, at about 45 degrees Celsius, or at a temperature in the range of 46-65 degrees Celsius, to remove most of the traces of liquid from the material. The dried material may be stored at about −20 degrees Celsius, or at approximately 4 degrees Celsius or at −70 to −80 degrees Celsius, before the next steps in the extraction process. Alternatively, the next steps in the extraction process may immediately commence. Of course, other suitable drying temperatures may be used.

Either before or after drying, the material from Spondias mombin L. may be ground to produce smaller particle sizes. In order to obtain approximately 20-50 micron particle size, the material from Spondias mombin L. may be ground using a suitable grinder or pulverizer, such as a Wiley mill rotary pulverizer, for example. In addition, filters may be used to separate out and obtain approximately 20-50 micron particle size. Thereafter and between the steps in the extraction process, the material from Spondias mombin L. may be stored at −20 degrees Celsius, or at approximately 4 degrees Celsius or at −70 to −80 degrees Celsius or other suitable temperatures, in substantially air tight plastic bags or other containers.

About 10-100 grams of material from Spondias mombin L. may be subjected to extraction using an extractor. Solvents of varying polarity may be used in connection with an extractor to extract and separate the various components from the material from Spondias mombin L., based on the polarity or solubility of the components. Initially, material from Spondias mombin L. may be placed inside a “thimble” made from filter paper. The thimble may be made of any suitable permeable material. The thimble with the material from Spondias mombin L. may be loaded into an extractor. The extractor may have a flask containing a solvent and a condenser. The solvent may be heated, which would cause the solvent to evaporate. The hot solvent vapor travels up to the condenser, where it cools and drips down into the chamber and onto the material from Spondias mombin L. Within the extractor, a chamber containing material from Spondias mombin L. slowly fills with warm solvent. At that point, components from the material are extracted from the material and form a component solution with the solvent. When the chamber is almost full, the component solution is emptied by siphon action, back down into the flask. During each cycle, components from the material from Spondias mombin L. are extracted into the solvent, resulting in a component solution. This cycle may be repeated many times with each solvent. During this extraction process, clean warm solvent may be used to extract components from the material from Spondias mombin L. in the thimble.

With respect to the solvents that may be used in connection with the extractor, a non-polar solvent such as Hexane-1 (“hexane”) or other non-polar solvents such as, but not limited to, Pentane, Cyclohexane, Heptane, Trichloroethylene, Carbon Tetrachloride, Diisopropyl Ether, or Toluene may be used. A moderately polar solvent such as Ethyl acetate-2 (“ethyl-acetate”) or other moderately polar solvents such as, but not limited to, Xylene, Methyl Butyl Ether, Diethyl Ether, Dichloromethane, Dichloroethane, n-Butanol, Isopropanol, Tetrahydrofuran, Butyl Acetate, Chloroform, n-Propanol, or Methyl Ethyl Ketone may be used. A polar solvent such as Methanol-3 (“methanol”) or other polar solvents such as, but not limited to, Acetone, Ethanol, Acetonitrile, Acetic Acid, Dimethyl Formamide, or Dimethyl Sulfoxide (DMSO) may be used. Extraction with the non-polar, moderately polar, and polar solvents may be performed at 45 degrees Celsius or other suitable temperatures, including but not limited to from approximately 26 degrees Celsius to approximately 60 degrees Celsius. Relatively pure water (“aqueous solvent”) may be used to extract components by soaking for approximately 12 hours, or between approximately 4 hours and 12 hours or other suitable times, the material from Spondias mombin L. which is remaining after using any of the polar, moderately polar, or non-polar solvents and filtering out the solid material, resulting in a component solution. Alternatively, material from Spondias mombin L. may be soaked in relatively pure water at any point during the extraction method or independent from treating the material with any solvent. As a control, periodically samples may be drawn and analyzed to evaluate the effect of exposure time on extraction.

Following the above process, the solvent which contains various components of Spondias mombin L., a component solution, is located in the flask of the extractor. Liquid may be at least partially removed by drying the component solution using a rotary evaporator or other suitable evaporator including, but not limited to, a vacuum drier, a vacuum oven, nitrogen gas, a thermofuel concentrator, a centrifuge and spray drier, or other suitable drying processes. This drying process may remove substantially all of the liquid from the component solution. The resulting extract may be frozen or freeze-dried. The extract may be stored in the form of an at least partially dry powder. The extract may be transferred to scintillation vials, which may be pre-weighed, and stored at −20 degrees Celsius or at approximately 4 degrees Celsius in a refrigerator or at −70 to −80 degrees Celsius or other suitable temperatures.

The result of the above described method, if hexane, ethyl-acetate, methanol and water are used, is four extracts of Spondias mombin L., with each extract containing components extracted by the solvent used. These extracts will be referred to as a hexane extract, an ethyl-acetate extract, a methanol extract and an aqueous extract (collectively, the “four extracts”).

Experimental Results

Experimental results demonstrate that an extract of Spondias mombin L. may be used to inhibit COX-2. Results described herein demonstrate that an extract of Spondias mombin L. decreases NF-Kappa B activation. Results described herein also demonstrate that an extract of Spondias mombin L. decreases the proliferation of SK-Mel 28 cells, a human melanoma cell line and decreases cell colony count in human breast cancer cell lines. In addition, an extract of Spondias mombin L. results in decreased growth in tumor volume in nude mice having tumors resulting from MCF-7 human breast cancer cells being implanted on the mammary fat pad.

An Extract of Spondias mombin L. and COX-2 Inhibition

An experiment was conducted that identified whether the extract of Spondias mombin L. at least partially inhibited COX-2 by assaying peroxidase activity of human recombinant COX-2 (the “COX-2 Inhibition Assay”). The COX-2 Inhibition Assay was performed on each of the four extracts.

The COX-2 Inhibition Assay was maintained and performed at approximately 37 degrees Celsius, by use of a water bath. Briefly, human recombinant COX-2 in reaction buffer (0.1 M Tris-HCl (pH 8.0), containing 5 mM EDTA and 2 mM phenol), heme, and arachidonic acid was incubated with each of the four extracts for two (2) minutes. The appearance of oxidized tetramethyl-p-phenyldiamine indicated the presence of peroxidase activity colorimetrically.

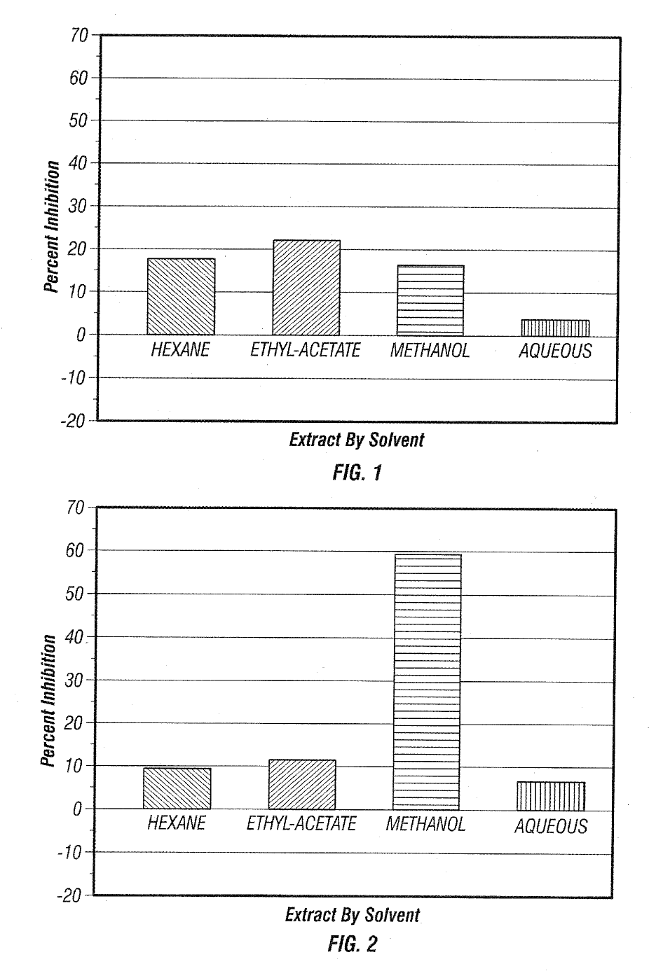

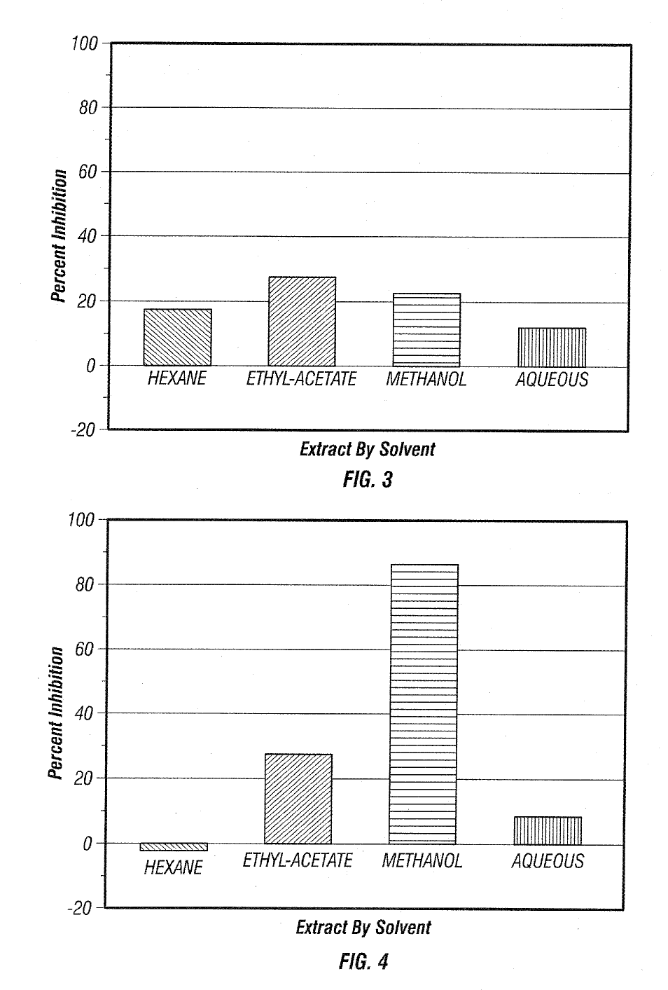

In preparation of the COX-2 Inhibition Assay, dried extracts were dissolved in methanol, Dimethyl sulfoxide (“DMSO”), or ethanol, and then diluted into the reaction buffer. The final concentration of the extracts was 9 μg/ml. 1M hydrochloric acid was added to stop COX-2 activity after a two (2) minute incubation. DUP-697, a known COX-2 inhibitor, was used as an internal control and, as expected, inhibited COX-2 with an IC50 of approximately 200 nM. The percentage inhibition of COX-2 was calculated by subtracting the quantified COX-2 activity of reactions with the extract from the quantified COX-2 activity of reactions without any COX-2 inhibitor and dividing the result by the quantified COX-2 activity of reaction without the extract. The percentage inhibition of COX-2 by the extracts ranged from 5% to 83%. The results demonstrated that the methanol extract may be more effective at inhibiting COX-2 than the ethyl-acetate extract, the hexane extract and the aqueous extract at this concentration. It is possible that an ethyl-acetate extract, a hexane extract or an aqueous extract may be more effective at inhibiting COX-2 at different concentrations. The COX-2 Inhibition Assay was conducted at least 4 times with the same variables. The results of the COX-2 Inhibition Assays described above are shown in FIGS. 1-4. Relative inhibition of COX-2 may involve the generation of an IC50 value.

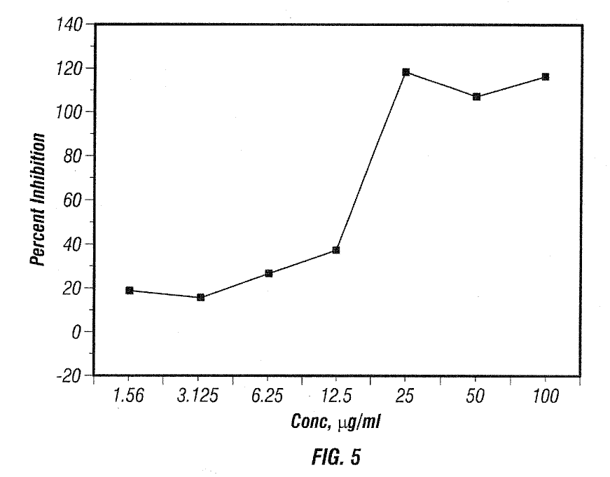

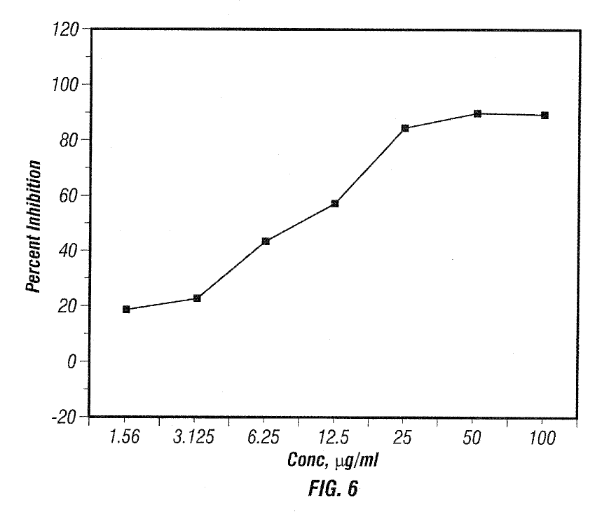

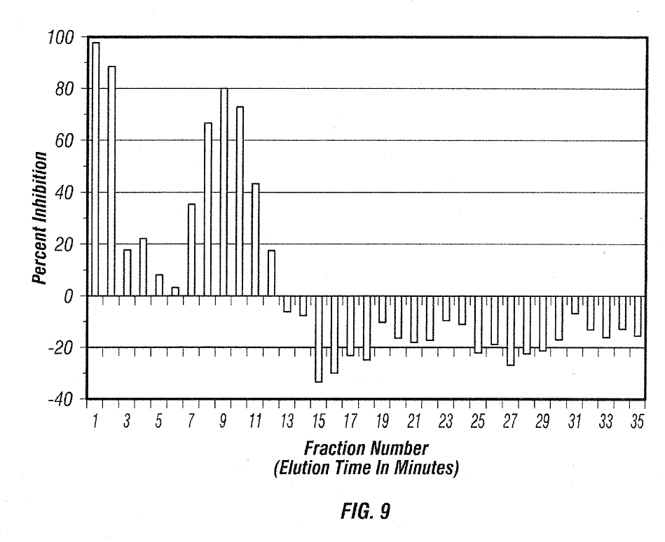

Additionally, a methanol extract was used in a COX-2 Inhibition Assay at different concentrations ranging from 1.56 μg/ml to 100 μg/ml. The results from two identical experiments are depicted on FIG. 5 and FIG. 6. The methanol extract showed an IC50 of 8.9 μg/ml in FIG. 5 and 9.0μ/ml in FIG. 6. A greater inhibition of COX-2 may be correlated with a higher concentration of the methanol extract.

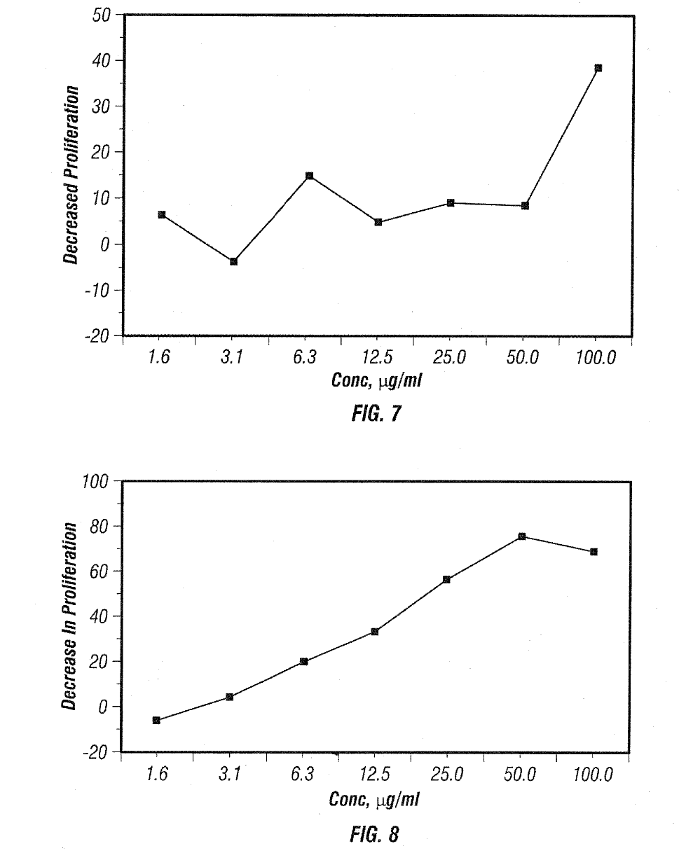

An additional assay was conducted to determine the effect of a methanol extract on the proliferation of human cancer cells. The human melanoma cell line, SK-Mel 28, was used in two independent experiments. A methanol extract was administered to the SK-Mel 28 cells in differing concentrations ranging from 1.6 μg/ml to 100 μg/ml. The methanol extract was administered to SK-Mel 28 cells cultured in the presence and absence of human growth serum, since serum, which normally contains various growth factors, may interfere in the inhibition of SK-Mel 28 cell proliferation. Cell proliferation was measured by the CellTiter 96 Aqueous One Solution Cell Proliferation Assay (Promega Corporation, Madison, Wis.) that uses a tetrazolium compound [3-(4,5-dimethylthiazol-2-yl)-5-(3-carboxymethoxyphenyl)-2-(4-sulfophenyl)-2H-tetrazolium, innersalt, or “MTS”], in combination with an electron coupling reagent (phenazine ethosulfate, or “PES”), to produce a calorimetric change indicating cell proliferation. The assay measured the decrease in proliferation of SK-Mel 28 by the methanol extract by recording absorbance at 490 nm. The results of the assay are shown in FIGS. 7 and 8. FIG. 7 shows that the methanol extract at 100 μg/ml produced about a 40% decrease of the proliferation of human melanoma cell line SK-Mel 28 in growth medium supplemented with serum, with an IC50 of over 100 μg/ml. The decrease in proliferation of SK-Mel 28 by the methanol extract was also assayed in SK-Mel 28 cells in growth medium without serum, the results of which are shown in FIG. 8. A pronounced decrease of SK-Mel 28 cell proliferation by the methanol extract with an IC50 of 75 μg/ml was observed as shown in FIG. 8. Also, FIGS. 7-8 illustrate results showing that the methanol extract decreased the proliferation of SK-Mel 28 in a dose-dependent manner.

In order to identify the components of a methanol extract responsible for inhibition of COX-2, a methanol extract from Spondias mombin L. was fractionated on a semi-preparative column. The methanol extract was loaded onto an Agilent™ Zorbax™ XDB C18 21.2×100 mm Column using a CTC Analytics™ PAL™ injector (liquid chromatography automated injector), with an injection volume of 50 μl. The methanol extract was eluted using a Shimadzu™ LC-6™ binary high pressure system. The first mobile phase was H₂O with 0.05% trifluoroacetic acid (“TFA”), and the second mobile phase was methanol with 0.05% TFA. A post-column split was employed having two Valco Y fittings with a 30 μm internal diameter (“id”) by 15 cm long restriction capillary and an approximate split ratio of 500:1. The fraction collector was an Advantec, with time set based on fractionation starting at 0.8 min, and 0.20 min collection steps. Of course, other variables may be used to accomplish the same or similar fractionation. Fractions eluting at different times may be collected, dried under nitrogen or freeze-dried, resulting in relatively pure fractions. The dried fractions may be incorporated into a medicament to treat any disease or ailment which may be treated by inhibiting COX-2, including but not limited to diseases related to inflammation or cancer, according to methods known in the art.

The inhibition of COX-2 by the fractions from the Spondias mombin L. methanol extract was examined by COX-2 Inhibition Assays. In order to prepare the dried fractions for a COX-2 Inhibition Assay, dried fractions were suspended in 100% DMSO and water for a concentration of 0.5% DMSO. A control with only 0.5% DMSO may be included in the COX-2 Inhibition Assay. The fractions were tested at varying concentrations such as, but not limited to, 1.56-100 μg/ml. The results of the COX-2 Inhibition Assay using the fractions from a Spondias mombin L. methanol extract, at a concentration of 10 μg/ml, are shown on FIG. 9. FIG. 9 shows fraction number by elution time in minutes on the x-axis (with elution time in minutes hereinafter being the identifying number of each fraction), and percentage inhibition of human recombinant COX-2 on the y-axis. As can be seen in FIG. 9, fractions 1 and 2, which eluted at one minute and two minutes, respectively, showed the most COX-2 inhibition, while fractions 8, 9 and 10 also showed significant COX-2 inhibition. Some inhibition was exhibited by fractions 3, 4, 5, 6, 7, 11, and 12.

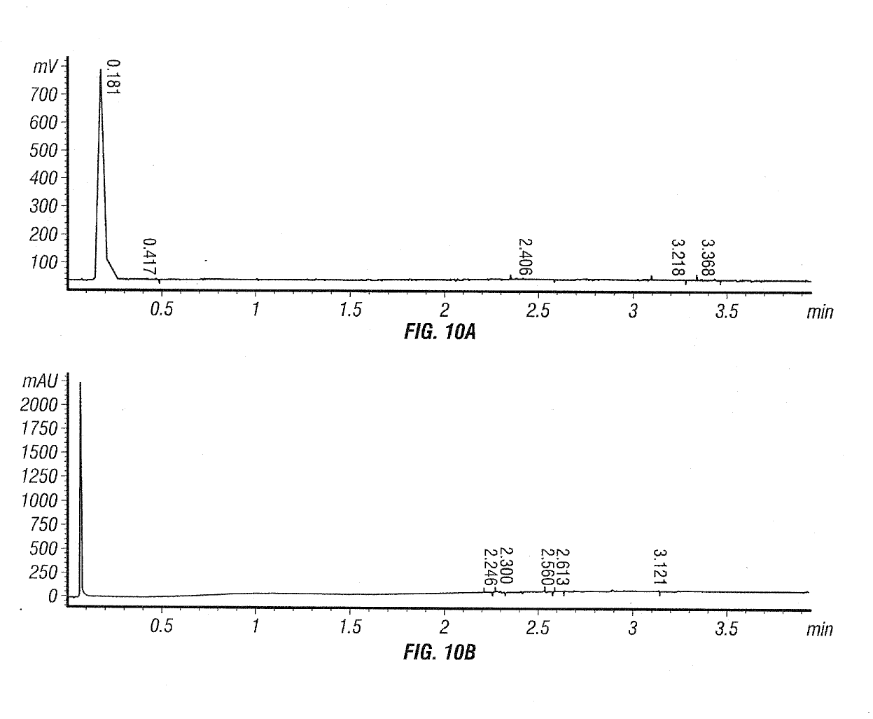

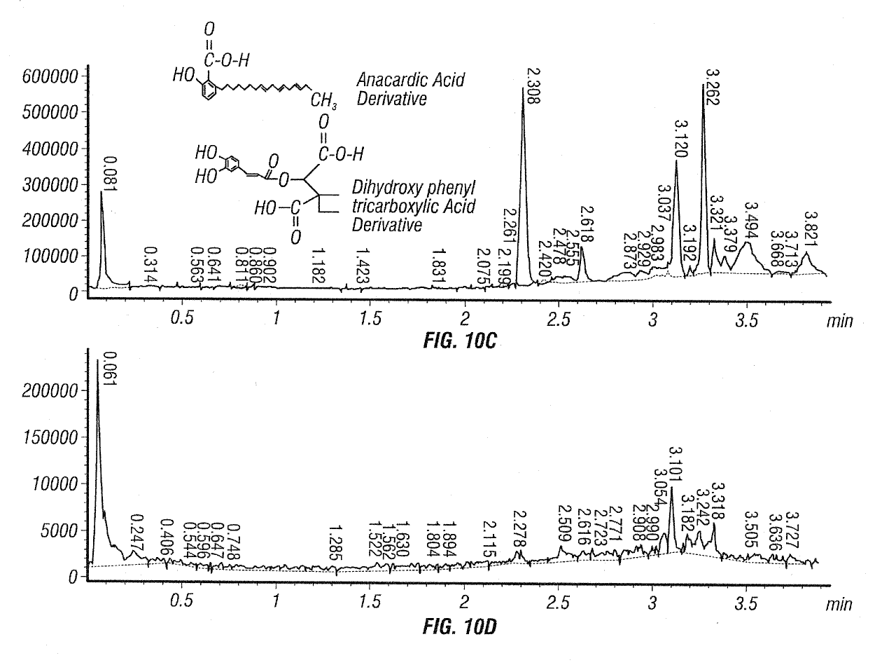

The fractions that showed the most COX-2 inhibition, specifically fractions 1 and 2, were profiled for structure using analytical Liquid Chromatography-Mass Spectrometry (“LC-MS”) on a hypersil C18 reverse phase column (100×2.1 mm, 5 mm) and eluted with a water-acetonitrile gradient on a flow rate of 0.6 ml/min. FIGS. 10A-10D show the mass spectra from fractions 1 and 2. As shown in FIGS. 10A-10D, components that may be present in fractions 1 and 2 include an Anacardic acid derivative or a Dihydroxy phenyltricarboxylic acid derivative.

An Extract of Spondias mombin L. and a NF-Kappa B Assay

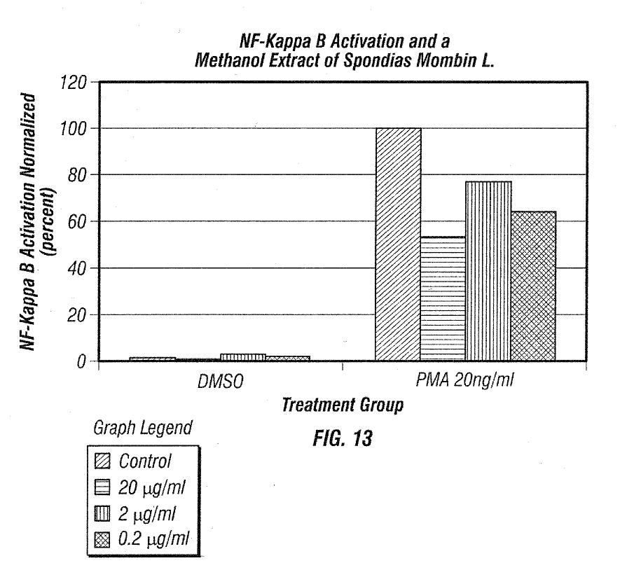

An NF-Kappa B assay demonstrated that a methanol extract of Spondias mombin L., an aqueous extract of Spondias mombin L. and a hexane extract of Spondias mombin L. decrease the inflammatory response in vitro in human embryonic kidney 293 cells. The results from an NF-Kappa B assay with an ethyl-acetate extract of Spondias mombin L. to test the inflammatory response in vitro in human embryonic kidney 293 cells were inconclusive. NF-Kappa B is a transcription factor. It is understood by persons of ordinary skill in the art that NF-Kappa B activation/expression is one of many early inflammatory responses and is an indicator of inflammation. Inhibition of NF-Kappa B activation may involve a corresponding inhibition of inflammation. Thus, an extract that inhibits NF-Kappa B activation may treat inflammation in a patient. FIGS. 13-20 show the results of an NF-Kappa B report gene assay to test the effects of a methanol extract of Spondias mombin L., an aqueous extract of Spondias mombin L. and a hexane extract of Spondias mombin L. on NF-Kappa B activation. Human embryonic kidney 293 cells were transfected with a DNA plasmid containing a NF-Kappa B response element upstream of the firefly luciferase gene. If NF-Kappa B is activated, there is an increased luciferase expression. Luciferase expression is measured by an enzyme reaction in which the luciferase produces light. A greater degree of light corresponds with an increased NF-Kappa B activation. Human embryonic kidney 293 cells were plated in charcoal stripped media overnight.

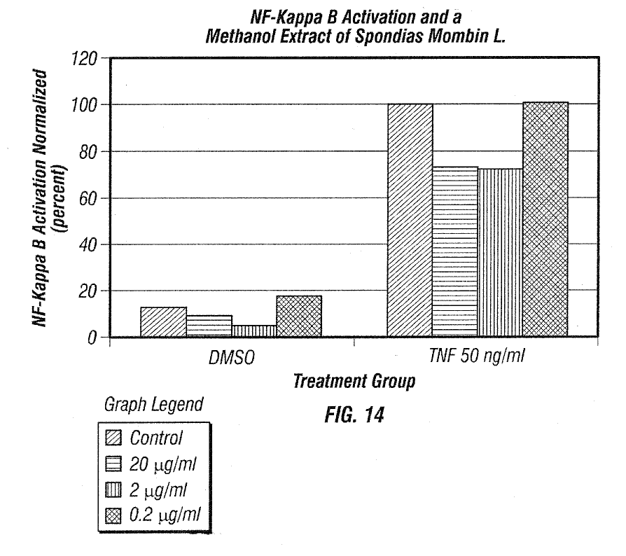

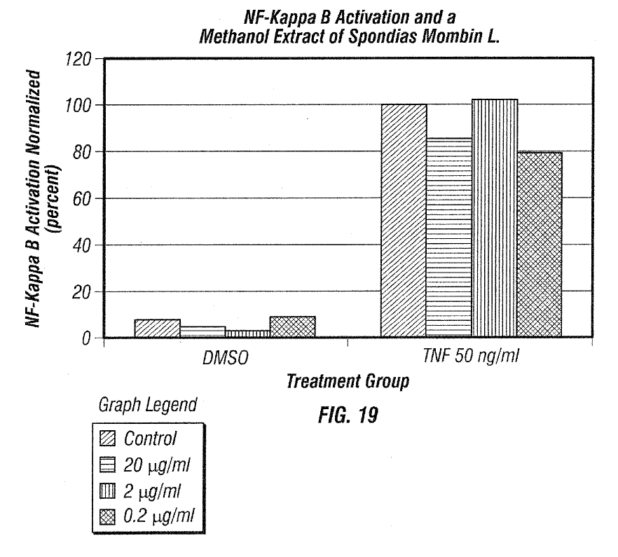

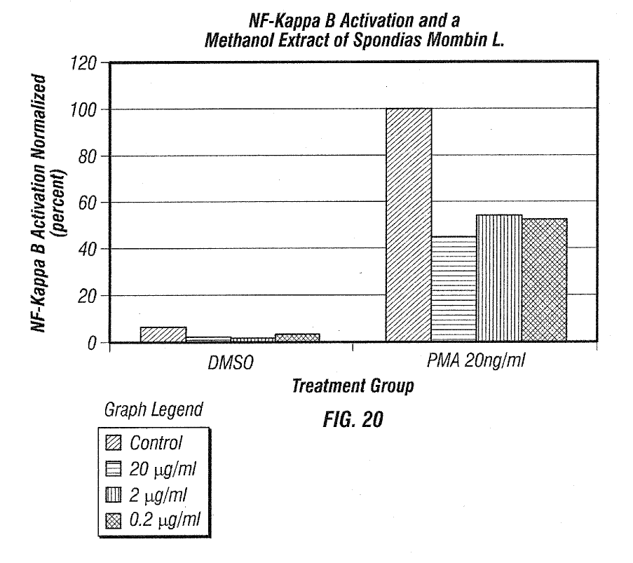

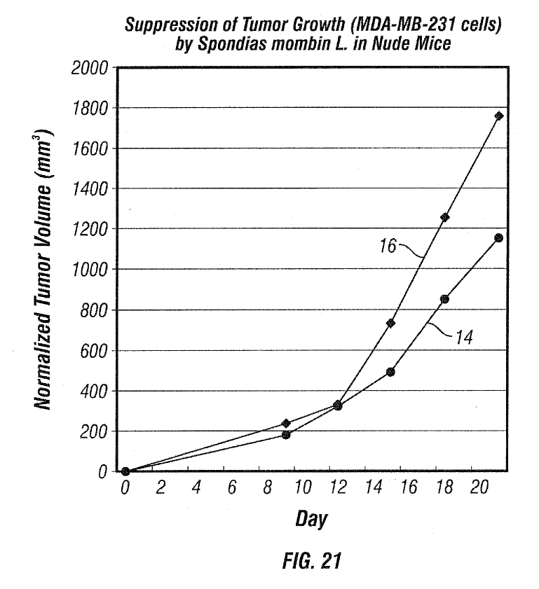

Both TNF (tumor necrosis factor) and PMA (phorbol ester) are known potent activators of NF-Kappa B. Human embryonic kidney 293 cells were exposed separately to DMSO only and PMA at a concentration of 20 ng/ml, and in a second experiment, DMSO only and TNF at a concentration of 50 ng/ml, and each of the foregoing treatment groups were given a dose range of a methanol extract of Spondias mombin L., an aqueous extract of Spondias mombin L., or a hexane extract of Spondias mombin L. dissolved in DMSO at 20 μg/ml, 2.0 μg/ml or 0.2 μg/ml and incubated overnight. The cells were harvested and lysed the following day for luciferase assay. The percent activation of NF-Kappa B remaining after each treatment was observed. The activation of NF-Kappa B was normalized to 100% with respect to the control containing DMSO and the known activator (TNF or PMA). The results from a NF-Kappa B assay using a methanol extract of Spondias mombin L. are depicted in FIGS. 13-14 and FIGS. 19-20. FIGS. 13 and 20 are graphs that illustrate the results from two separate experiments to test NF-Kappa B activation in cells treated with PMA 20 ng/ml and a methanol extract of Spondias mombin L. As illustrated in FIG. 13, in the PMA 20 ng/ml treatment group, NF-Kappa B activation was approximately 53% in cells treated with a methanol extract of Spondias mombin L. at 20 μg/ml, approximately 77% in cells treated with a methanol extract of Spondias mombin L. at 2 μg/ml, and approximately 63% in cells treated with a methanol extract of Spondias mombin L. at 0.2 μg/ml. In FIG. 20, in the PMA 20 ng/ml treatment group, NF-Kappa B activation was approximately 44% in cells treated with a methanol extract of Spondias mombin L. at 20 μg/ml, approximately 54% in cells treated with a methanol extract of Spondias mombin L. at 2 μg/ml, and approximately 53% in cells treated with a methanol extract of Spondias mombin L. at 0.2 μg/ml. FIGS. 14 and 19 are graphs that illustrate the results from two separate experiments to test NF-Kappa B activation in cells treated with TNF 50 ng/ml and a methanol extract of Spondias mombin L. As illustrated in FIG. 14, in the TNF 50 ng/ml treatment group, NF-Kappa B activation was approximately 72% in cells treated with a methanol extract of Spondias mombin L. at 20 g/ml and approximately 71% in cells treated with a methanol extract of Spondias mombin L. at 2 μg/ml, and approximately 101% in cells treated with a methanol extract of Spondias mombin L. at 0.2 μg/ml. In FIG. 19, in the TNF 50 ng/ml treatment group, NF-Kappa B activation was approximately 85% in cells treated with a methanol extract of Spondias mombin L. at 20 μg/ml, approximately 102% in cells treated with a methanol extract of Spondias mombin L. at 2 μg/ml, and approximately 79% in cells treated with a methanol extract of Spondias mombin L. at 0.2 μg/ml.

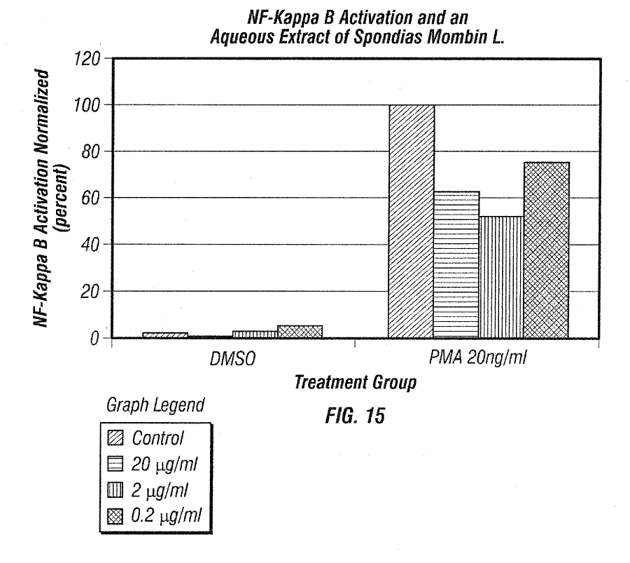

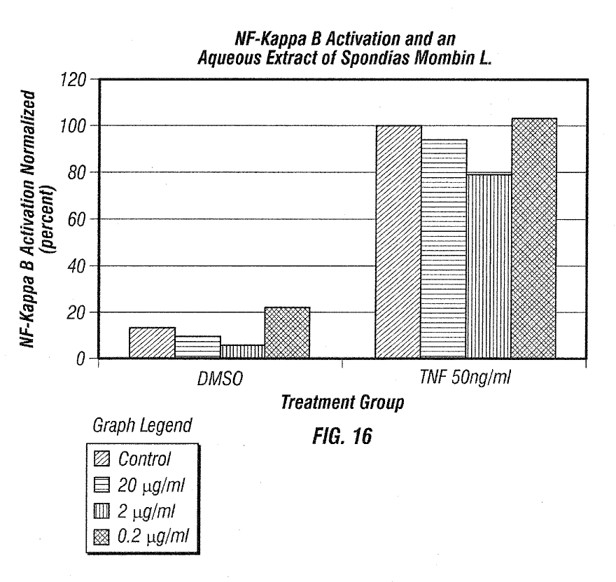

The results from the NF-Kappa B assay using an aqueous extract of Spondias mombin L. are depicted in FIGS. 15-16. FIG. 15 is a graph that illustrates the results from an experiment to test NF-Kappa B activation in cells treated with PMA 20 ng/ml and an aqueous extract of Spondias mombin L. As illustrated in FIG. 15, in the PMA 20 ng/ml treatment group, NF-Kappa B activation was approximately 63% in cells treated with an aqueous extract of Spondias mombin L. at 20 μg/ml, approximately 50% in cells treated with an aqueous extract of Spondias mombin L. at 2 μg/ml, and approximately 74% in cells treated with an aqueous extract of Spondias mombin L. at 0.2 μg/ml. FIG. 16 is a graph that illustrates the results from an experiment to test NF-Kappa B activation in cells treated with TNF 50 ng/ml and an aqueous extract of Spondias mombin L. In the TNF 50 ng/ml treatment group, NF-Kappa B activation was approximately 94% in cells treated with an aqueous extract of Spondias mombin L. at 20 μg/ml, approximately 78% in cells treated with an aqueous extract of Spondias mombin L. at 2 μg/ml, and approximately 103% in cells treated with an aqueous extract of Spondias mombin L. at 0.2 μg/ml.

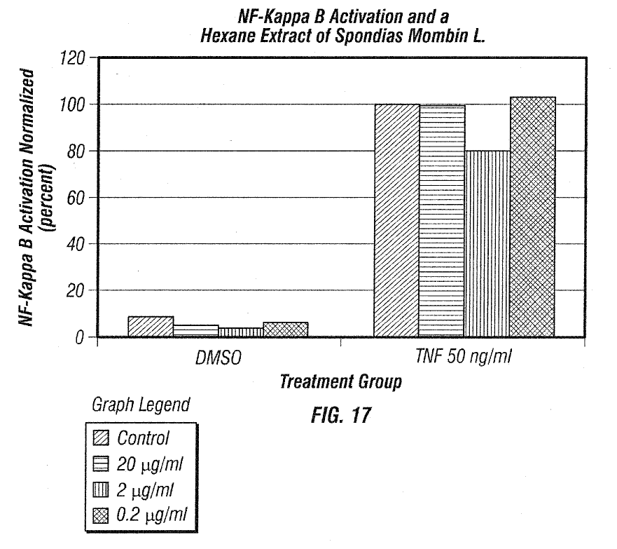

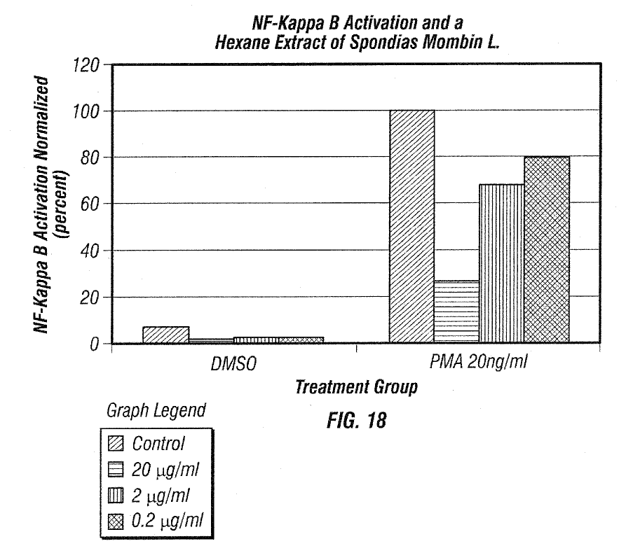

The results from the NF-Kappa B assay using a hexane extract of Spondias mombin L. are depicted in FIGS. 17-18. FIG. 17 is a graph that illustrates the results from an experiment to test NF-Kappa B activation in cells treated with TNF 50 ng/ml and a hexane extract of Spondias mombin L. As illustrated in FIG. 17, in the TNF 50 ng/ml treatment group, NF-Kappa B activation was approximately 99% in cells treated with a hexane extract of Spondias mombin L. at 20 μg/ml, approximately 80% in cells treated with a hexane extract of Spondias mombin L. at 2 μg/ml, and approximately 103% in cells treated with a hexane extract of Spondias mombin L. at 0.2 μg/ml. FIG. 18 is a graph that illustrates the results from an experiment to test NF-Kappa B activation in cells treated with PMA 20 ng/ml and a hexane extract of Spondias mombin L. As illustrated in FIG. 18, in the PMA 20 ng/ml treatment group, NF-Kappa B activation was approximately 27% in cells treated with a hexane extract of Spondias mombin L. at 20 μg/ml, approximately 68% in cells treated with a hexane extract of Spondias mombin L. at 2 μg/ml, and approximately 80% in cells treated with a hexane extract of Spondias mombin L. at 0.2 μg/ml.

An Extract of Spondias mombin L. and Growth of Cancer

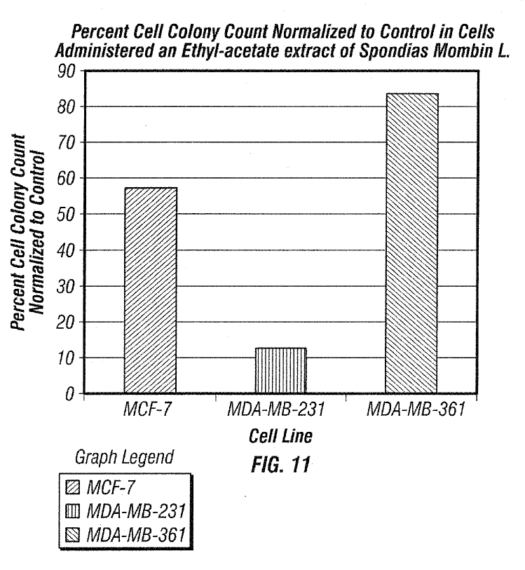

An in vitro cell culture assay demonstrated that an ethyl-acetate extract of Spondias mombin L. decreases the growth of cell colony formation in cancer cell lines. The results from an in vitro cell culture assay with an aqueous extract of Spondias mombin L., a hexane extract of Spondias mombin L, and a methanol extract of Spondias mombin L. were inconclusive. FIG. 11 depicts results using three breast cancer cell lines: MCF-7 (an estrogen receptor positive human breast cancer cell line), human breast cancer cell line MDA-MB-231 (an estrogen independent cancer cell line that originated from a human metastatic ductal breast carcinoma sample), and MDA-MB-361 (an estrogen receptor positive human breast cancer cell line derived from cerebral metastatic tissue).

Cells of the human breast cancer cell lines MCF-7, MDA-MB-231, MDA-MB-361 were seeded onto culture plates in media supplemented with serum. The cells were exposed to an ethyl-acetate extract of Spondias mombin L. dissolved in DMSO at a concentration of 10 μg/ml. A 7-10 day growth assay was performed. Cell growth was monitored by counting the cell colonies by staining the colonies and manually counting, with 50 cells equaling one colony. In the graph in FIG. 11, the results represent the number of cells that survive early exposure to an ethyl-acetate extract of Spondias mombin L. and grow to form visible colonies. The raw colony count (number of colonies) for each cell line was normalized with the control for that treatment group (100%). The normalized cell colony count for each cell line exposed to an ethyl-acetate extract of Spondias mombin L. is represented in FIG. 11, where each bar is the average of duplicate samples in that particular cell line exposed to an ethyl-acetate extract of Spondias mombin L. The graph in FIG. 11 comprises cell line on the x-axis and cell colony count normalized with the control to 100% on the y-axis. As shown in FIG. 11, the ethyl-acetate extract of Spondias mombin L. inhibited the growth of MDA-MB-231 to the greatest extent as compared to the other cell lines, showing approximately 12.7% normalized cell colony count, with the growth of MCF-7 having a normalized cell colony count of approximately 57.2%, and MDA-MB-361 having a normalized cell colony count of approximately 83%.

Experimental results demonstrate that an ethyl-acetate extract of Spondias mombin L. may suppress breast cancer tumor growth in vivo. Ten female nude mice were injected with 100 μl Reduced Growth Factor Matrigel from BD Biosciences™ and 5×10⁶ MCF-7 cells (estrogen receptor positive human breast carcinoma cells) on the mammary fat pad and ten female nude mice were injected with 100 μl Reduced Growth Factor Matrigel from BD Biosciences™ and 5×10⁶ MDA-MB-231 at two subcutaneous dorsal sites, with the foregoing all suspended in 50 μl Phosphate Buffered Saline (“PBS”). Following tumor formation, which occurred at approximately 10 days post injection for mice injected with MDA-MB-231 cells and 15 days post injection for mice injected with MCF-7 cells, the nude mice were randomized into treatment groups. One group of nude mice were injected intraperitoneally (“IP”) daily with 20 mg/kg/mouse of an ethyl-acetate extract of Spondias mombin L. suspended in a 50 μl 1:5 DMSO:PBS solution with a final concentration of 8 mg/ml. Prior to suspension in the DMSO:PBS solution, the dried ethyl-acetate extract was re-suspended in pure ethanol, aliquoted, and the ethanol was allowed to evaporate, leaving a dry powder. A control group of nude mice were treated with the 1:5 DMSO:PBS solution (“control”) only. The nude mice were administered either the control or an ethyl-acetate extract of Spondias mombin L. Tumors were measured every other day. Tumor volume was calculated by measuring the short and long axis of the tumor with calipers in millimeters, and using the equation 4.19×(Long axis/2)×(short axis/2)₂ to arrive at tumor volume in cubic millimeters (mm³). The tumor volume values were normalized.

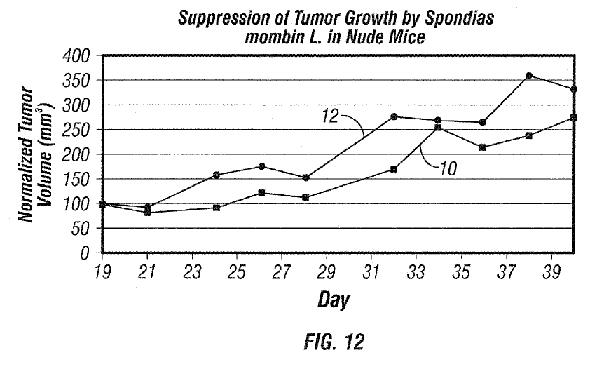

The results from two experiments using the foregoing methods are depicted in FIGS. 12 and 21, with normalized tumor volume on the y-axis in mm³ and day post-injection of MDA-MB-231 cells or MCF-7 cells on the x-axis. Mice were injected on their mammary fat pad with MCF-7 cells in the experiment depicted in FIG. 12, with 5 mice in the control group and 5 mice in the group administered an ethyl-acetate extract of Spondias mombin L. at a final concentration of 8 mg/ml, each for 14 consecutive days. Tumors were measured every other day starting 15 days post injection. Plot line 10 represents the results from 5 nude mice that were administered an ethyl-acetate extract of Spondias mombin L., and plot line 12 represents the results from 5 nude mice that were administered the control. In the experiment depicted in FIG. 21, 10 female nude mice were injected with MDA-MB-231 cells at two subcutaneous dorsal sites. Plot line 14 represents the results from 5 nude mice that were administered an ethyl-acetate extract of Spondias mombin L. at a concentration of 8 mg/ml, and plot line 16 represents the results from 5 nude mice that were administered the control, each starting at 10 days post injection. As shown in FIGS. 12 and 21, an ethyl-acetate extract of Spondias mombin L. suppressed tumor growth as compared to the control.

It is understood by a person of ordinary skill in the art that a component that treats a condition by intraperitoneal injection is likely to treat a condition if administered parenterally to a patient. An extract of Spondias mombin L. may be administered to a patient parenterally to treat breast cancer.

An extract of Spondias mombin L. may be created using the methods described herein. Methanol or some other solvent, such as but not limited to a polar, non-polar, moderately polar or aqueous solvent, may be used to extract the components of Spondias mombin L. that at least partially inhibit COX-2. It is understood by persons of ordinary skill in the art that inhibiting COX-2 results in a decrease in inflammation, as COX-2 is an enzyme that contributes to the immune response generally referred to as inflammation. It is also understood by persons of ordinary skill in the art that inhibiting COX-2 results in apoptosis of cancer cells or a decrease in the proliferation of cancer cells. As such, an extract of Spondias mombin L. may be incorporated into a medicament and would be expected to treat cancer by either decreasing the proliferation of cancer cells or inducing the apoptosis of cancer cells. Such an extract may also be concentrated or dried and incorporated into a medicament. Alternatively, such an extract may be fractionated in order to further isolate certain fractions that inhibit COX-2. For example and without limitation, fractions 1-2 and fractions 8, 9, and 10, in any combination or individually, may be incorporated into a medicament to at least partially inhibit COX-2 in patients.

It is expected that a medicament containing an extract of Spondias mombin L. may be administered in a therapeutically effective amount to a patient to treat inflammation or to treat cancer. A medicament may be prepared containing an extract of Spondias mombin L. and formulated to administer to a patient by procedures known by a person of ordinary skill in the art.

A medicament containing an extract of Spondias mombin L. may be prepared by conventional procedures, known by a person of ordinary skill in the art, for blending and mixing compounds. For example, a methanol extract or an ethyl-acetate extract of Spondias mombin L., or fractions from a methanol extract or an ethyl-acetate extract of Spondias mombin L. such as fractions 1-2 or fractions 8, 9, and 10 alone or in combination, may be formulated in a therapeutically effective amount into a solution, a suspension, a powder, a capsule, a tablet, or a liquid, by use of a pharmaceutically acceptable vehicle to facilitate oral or enteral administration of the extract to treat a patient. Alternatively, an extract of Spondias mombin L. or particular fractions of an extract of Spondias mombin L. may be incorporated into a pharmaceutically acceptable vehicle to facilitate parenteral administration. In an alternative embodiment, an extract from Spondias mombin L. or fractions from an extract of Spondias mombin L., alone or in combination, may be incorporated into a solution, cream, or gel using a pharmaceutically acceptable vehicle for topical application, or transdermal application.

Although the foregoing specific details describe certain embodiments of this invention, persons reasonably skilled in the art will recognize that various changes may be made in the details of this invention without departing from the spirit and scope of the invention as defined in the appended claims and considering the doctrine of equivalents. Therefore, it should be understood that this invention is not to be limited to the specific details shown and described herein.

What is claimed is:

1. A method of inhibiting COX-2 comprising:
administering a therapeutically effective amount of an extract of Spondias mombin L. to a patient.

2. The method of claim 1 wherein said extract is selected from the group consisting of:
a methanol extract;
an ethyl-acetate extract;
a hexane extract; and
an aqueous extract.

3. A method of treating inflammation comprising:
administering a therapeutically effective amount of an extract of Spondias mombin L. to a patient.

4. The method of claim 3 wherein said extract is selected from the group consisting of:
a methanol extract;
an ethyl-acetate extract;
a hexane extract; and
an aqueous extract.

5. A method of treating cancer comprising:
administering a therapeutically effective amount of an extract of Spondias mombin L. to a patient.

6. The method of claim 5 wherein said extract said extract is selected from the group consisting of:
a methanol extract;
an ethyl-acetate extract;
a hexane extract; and
an aqueous extract.

7. A medicament comprising:
a pharmaceutically acceptable vehicle; and
a therapeutically effective amount of an extract of Spondias mombin L. suspended in said vehicle.

8. An extract of Spondias mombin L. comprising components extracted using a solvent selected from the group consisting of:
a polar solvent;
a non-polar solvent;
a moderately polar solvent; and
an aqueous solvent.

9. A method of making an extract of Spondias mombin L. comprising:
creating a component solution by processing Spondias mombin L. material with an extractor and a solvent; and
producing an extract by at least partially removing liquid from said component solution.

10. The method of claim 9 wherein said solvent is selected from the group consisting of:
a polar solvent;
a non-polar solvent;
a moderately polar solvent; and
an aqueous solvent.

11. The method of claim 10 wherein said solvent is selected from the group consisting of:
methanol;
ethyl-acetate;
hexane; and
water.

12. The method of claim 10 further comprising:
obtaining at least one fraction of said extract by fractionating said extract.

13. The method of claim 10 further comprising:
obtaining at least one fraction of said extract by fractionating said extract on a semi-preparative column.

14. The method of claim 13 wherein said solvent comprises methanol and wherein said at least one fraction has an elution time in minutes selected from the group consisting of 1, 2, 3, 4, 5, 6, 7, 8, 9, 10, 11 and 12.

15. A method of making an extract of Spondias mombin L. comprising:
drying Spondias mombin L. material;
grinding said material;
creating a component solution by processing said material with an extractor and a solvent; and
producing an extract by at least partially removing liquid from said component solution.

16. A method of treating breast cancer comprising:
administering a therapeutically effective amount of an extract of Spondias mombin L. to a patient.

17. The method of claim 16 wherein said extract is selected from the group consisting of:
a methanol extract;
an ethyl-acetate extract;
a hexane extract; and
an aqueous extract.

18. The method of claim 17 wherein said therapeutically effective amount of said extract of Spondias mombin L. is administered parenterally in a pharmaceutically acceptable vehicle.

19. A method of inhibiting NF-Kappa B activation comprising:
administering a therapeutically effective amount of an extract of Spondias mombin L. to a patient

20. The method of claim 19 wherein said extract comprises a hexane extract.

21. The method of claim 19 wherein said extract comprises a methanol extract.

22. The method of claim 19 wherein said extract comprises an aqueous extract.
